(19) United States
(12) Patent Application Publication
(10) Pub. No.: US 2019/0175201 A1
(43) Pub. Date: Jun. 13, 2019

(54) ACOUSTIC GEL CONTAINER

(71) Applicant: i3 Ventures LLC, Indianapolis, IN (US)

(72) Inventors: Jeffery C. Smith, Poland, IN (US); John R. Lingeman, Plainfield, IN (US); Christopher W. Green, Bloomington, IN (US)

(73) Assignee: i3Ventures LLC, Indianapolis, IN (US)

(21) Appl. No.: 16/109,642

(22) Filed: Aug. 22, 2018

Related U.S. Application Data
(60) Provisional application No. 62/596,390, filed on Dec. 8, 2017.

(51) Int. Cl. A61B 17/225; A61J 1/14

(52) U.S. Cl. CPC A61B 17/2251; A61B 2017/2253; A61J 1/1412

(57) ABSTRACT

An acoustic gel pack includes a container, a first pad, and a second pad. The container is adapted to contain acoustic gel and includes a wall, a first end having a first opening, and a second end having a second opening. A chamber is defined within the wall between the first end and the second end. The first pad is removeably coupled to the container to seal the first opening. The second pad is removeably coupled to the container to seal the second opening. The seals formed by the first pad over the first opening and the second pad over the second opening are adapted to prevent fluid infiltration into the chamber.

### TECHNICAL FIELD

This disclosure relates to acoustic lithotripsy devices and in particular, to acoustic gel containers to be used with acoustic lithotripsy devices.

### BACKGROUND

The statements in this section merely provide background information related to the present disclosure and may not constitute prior art.

Acoustic lithotripsy is used to break up or move stones within the body of a patient. Examples of acoustic lithotripsy devices may include ultrasound or shockwave. The lithotripsy devices transmit concentrated sound waves through the body to break up the stone. The effectiveness of acoustic lithotripsy is dependent on the sound waves being able to travel through a consistent medium to reach the stones such that the speed of sound through the medium is consistent. Acoustic gel and other liquids are sometimes used as a coupling agent between the lithotripsy device to the body of the patient, providing a consistent medium between lithotripsy device and the body of the patient. However, air bubbles and impurities in the coupling agent can diminish and/or interfere with the uniform propagation of the sound waves through the medium, decreasing the effectiveness of the lithotripsy procedure. Therefore, a container which can be used to provide a consistent and effective coupling agent for acoustic lithotripsy is desirable.

### SUMMARY

Further areas of applicability will become apparent from the description provided herein. It should be understood that the description and specific examples are intended for purposes of illustration only and are not intended to limit the scope of the present disclosure.

In one embodiment, an acoustic gel pack is provided including a container, a first pad, and a second pad. The container includes a wall, a first end having a first opening, and a second end having a second opening. A chamber is defined within the wall between the first end and the second end. The first pad is removeably coupled to the container to seal the first opening. The second pad is removeably coupled to the container to seal the second opening. The seals formed by the first pad over the first opening and the second pad over the second opening are adapted to prevent fluid infiltration into the chamber.

In another embodiment, a lithotripsy system is provided including an acoustic wave generating apparatus, a container, a first pad, and a second pad. The acoustic wave generating apparatus includes an acoustic wave head. The container includes a wall having a first opening and a second opening. A chamber is defined within the wall between the first opening and the second opening. The chamber contains acoustic gel. The first pad is removeably coupled to the container to seal the first opening. The second pad is removeably coupled to the container to seal the second opening.

In yet another embodiment, a method of performing lithotripsy using an acoustic gel pack is provided including removing a first pad from a first opening of the acoustic gel pack, applying the first opening of the acoustic gel pack to one of an acoustic wave head or a body of a patient, removing a second pad from a second opening of the acoustic gel pack, and applying the second opening of the acoustic gel pack to the other of the acoustic wave head or the body of the patient.

### BRIEF DESCRIPTION OF THE DRAWINGS

The embodiments may be better understood with reference to the following drawings and description. The components in the figures are not necessarily to scale.

FIG. 1 illustrates a cross-sectional side view of a first example of an acoustic gel pack;

FIG. 2 illustrates a cross-sectional top-down view of a second example of the acoustic gel pack of FIG. 1;

FIG. 3 illustrates a perspective view of a third example of an acoustic gel pack;

FIG. 4 illustrates a cross-sectional side view of a fourth example of an acoustic gel pack;

FIG. 5 illustrates a cross-sectional side view of a fifth example of an acoustic gel pack;

FIG. 6 illustrates a cross-sectional side view of a sixth example of an acoustic gel pack;

FIG. 7 illustrates a cross-sectional side view of a seventh example of an acoustic gel pack;

FIG. 8 illustrates a cross-sectional side view of two eighth examples of acoustic gel packs;

FIG. 9 illustrates a cross-sectional front view of a ninth example of an acoustic gel pack;

FIG. 10 illustrates a cross-sectional side view of the ninth example of the acoustic gel pack of FIG. 9;

FIG. 11 illustrates a side view of a first step in an example method of performing lithotripsy including an acoustic gel pack, a lithotripsy device, and a body;

FIG. 12 illustrates a side view of a second step in an example method of performing lithotripsy including an acoustic gel pack, a lithotripsy device, and a body;

FIG. 13 illustrates a side view of a third step in an example method of performing lithotripsy including an acoustic gel pack, a lithotripsy device, and a body; and

FIG. 14 illustrates a flow diagram of example operations to perform as part of a lithotripsy procedure.

The drawings described herein are for illustration purposes only and are not intended to limit the scope of the present disclosure in any way. The various descriptors provided for the elements are examples and should not be construed as describing the extent of the features/functionality and therefore should not be construed as limiting in this regard.

### DETAILED DESCRIPTION

The following description is merely exemplary in nature and is not intended to limit the present disclosure, application, or uses.

In one example, an acoustic gel pack is provided including a container, a first pad, and a second pad. The container includes a wall, a first end having a first opening, and a second end having a second opening. A chamber is defined within the wall between the first end and the second end. The first pad is removeably coupled to the container to seal the first opening. The second pad is removeably coupled to the container to seal the second opening. The seals formed by the first pad over the first opening and the second pad over the second opening are adapted to prevent fluid infiltration into the chamber.

One interesting feature of the systems and methods described below may be that the acoustic gel pack may provide a sealed environment preventing infiltration of gas and other contaminants into the acoustic gel prior to use. Another feature may be that the acoustic gel pack may be easy to fill from a vacuum state with gas-evacuated acoustic gel. Yet another feature may be that the acoustic gel pack may be easy to unseal and apply to the lithotripsy device and to the body of a patient. Yet another feature may be that the acoustic gel pack may promoted purging of gas bubbles during the process of coupling the lithotripsy device to the body of the patient. Yet another feature may be that the container may maintain coupling while allowing for adjustment of standoff distance between an acoustic wave head and the body of the patient, ensuring that the acoustic wave is focused at an appropriate depth.

In addition to the features that have been explicitly described, there are other features and advantages that are depicted and represent part of the system. While various embodiments have been described, it will be apparent to those of ordinary skill in the art that many more embodiments and implementations are possible. Accordingly, the embodiments described herein are examples, not the only possible embodiments and implementations.

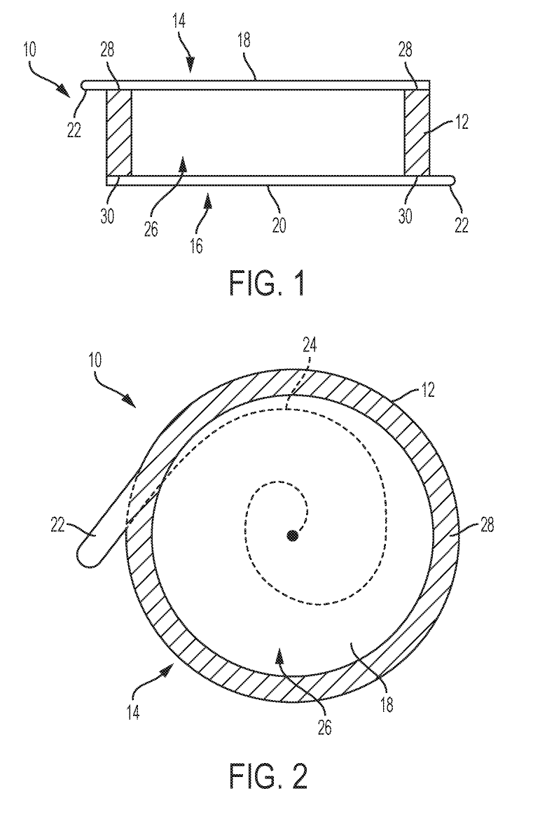

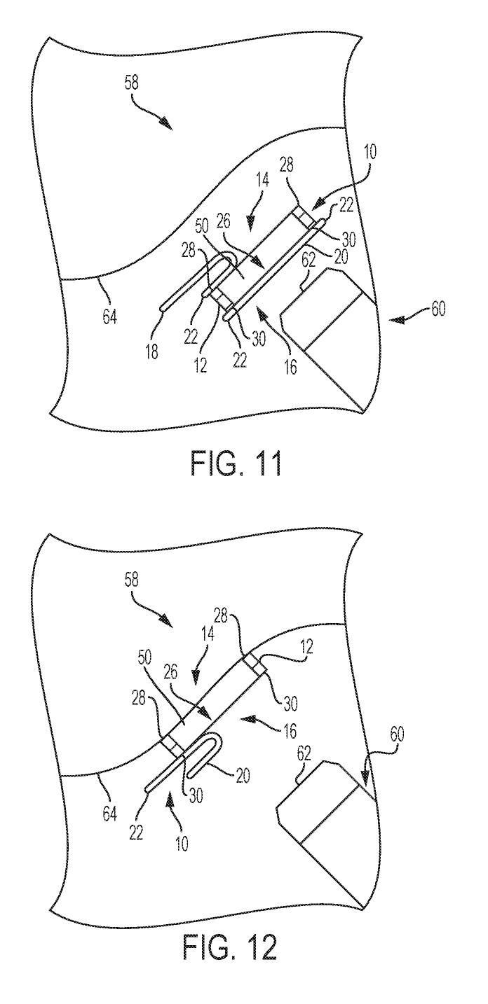

FIG. 1 illustrates an acoustic gel pack 10 including a wall 12 extending from a first surface 28 to a second surface 30, a first pad 18 coupled to the first surface 28 of the wall 12, and a second pad 20 coupled to the second surface 30 of the wall 12. The acoustic gel pack 10 may be any device which can be used to contain and apply acoustic gel (50 in FIG. 11) to an operating surface (64 in FIG. 11) of a patient (58 in FIG. 11). Examples of the acoustic gel pack 10 may include a container, a bag, or a canister. The acoustic gel pack 10 may be used in a variety of procedures, including extra-corporeal shockwave lithotripsy, burst wave lithotripsy, high intensity focused ultrasound probes, tissue ablations, and other non-stone breaking therapies. Some examples of non-stone breaking therapies in which the acoustic gel pack 10 may be used may include extra-corporeal shock wave for myocardial revascularization, radial shockwave therapy for tendinosis, bursitis, epicondylitis, tendonitis, fasciitis, trigger finger, back pain, muscle-knots, non-union fractures, avascular necrosis, stress fracture, cellulite and body firming, prostate tissue ablation, equine orthopedic therapy, prostate, kidney, liver, pancreatic, bladder, and other cancers, uterine fibroids, neurological disorders, cosmetic therapies, thyroid nodule treatment, and breast fibromyalgia treatment.

The wall 12 may be any structure configured to contain an acoustic gel 50 and which is configured to prevent infiltration of gases such as air. Examples of the wall 12 may include a shell, a cylindrical barrier, or a bag. The wall 12 may define a first opening 14 at the first end of the wall 12 and may define a second opening 16 at the second end of the wall 12. A chamber 26 may be enclosed within the wall 12, the chamber 26 extending from the first opening 14 to the second opening 16. The wall 12 may be sufficiently rigid to contain the acoustic gel 50 within the chamber 26 but may also be flexible enough to be removed from the acoustic gel 50 once the acoustic gel 50 has been applied to an operating surface 64. In some embodiments, the wall 12 may include a break, allowing the wall 12 to be removed by unwrapping the wall 12 about the chamber 26. The wall 12 may be made of materials such as polymers, rubber, or aluminum.

The first pad 18 may be any structure which may be applied to the first opening 14 of the wall 12. Examples of the first pad 18 may include a covering, a film, or a sheet. The first pad 18 may completely extend over the first opening 14 to prevent the acoustic gel 50 from escaping from the chamber 26 through the first opening 14. The first pad 18 may also seal the first opening 14 to prevent gas from infiltrating the acoustic gel 50 within the chamber 26. The first pad 18 may be coupled to a first surface 28 at the first end of the wall 12 by an adhesive. The first pad 18 may be removeable from the first opening 14 by peeling the first pad 18 from the first surface 28 of the wall 12 or by puncturing the first pad 18. The first pad 18 may be made of any material which may maintain a seal over the first opening 14 such as a polymer film. The first pad 18 may be transparent to allow inspection of the acoustic gel 50 within the chamber 26 to ensure that any gas contained within the acoustic gel 50 in the chamber 26 is at an acceptably low level.

The second pad 20 may be any structure which may be applied to the second opening 16 of the wall 12. Examples of the second pad 20 may include a covering, a film, or a sheet. The second pad 20 may completely extend over the second opening 16 to prevent the acoustic gel 50 from escaping from the chamber 26 through the second opening 16. The second pad 20 may also seal the second opening 16 to prevent gas from infiltrating the acoustic gel 50 within the chamber 26. The second pad 20 may be coupled to a second surface 30 at the second end of the wall 12 by an adhesive. The second pad 20 may be removeable from the second opening 16 by peeling the second pad 20 from the second surface 30 of the wall 12 or by puncturing the second pad 20. The second pad 20 may be made of any material which may maintain a seal over the second opening 16 such as a polymer film. The second pad 20 may be transparent to allow inspection of the acoustic gel 50 within the chamber 26 to ensure that any gas contained within the acoustic gel 50 in the chamber 26 is at an acceptably low level. The first pad 18 and the second pad 20 may be a circular, may be oval-shaped, or may be any other shape which may seal the first opening 14 or second opening 16.

The first pad 18 and second pad 20 may each also include a tab 22. The tab 22 may extend over the wall 12 of the acoustic gel pack 10 such that the tab 22 may be gripped by fingers of a user. Examples of the tab 22 may include a lip, a flap, or an overhanging projection. The tab 22 may be textured to allow gripability by the user.

The acoustic gel 50 may be any solution through which acoustic energy may be transmitted with minimal loss. Properties relating to wave transmission may include sound velocity, acoustic impedance, and sound attenuation. Examples of acoustic gel 50 may include low viscosity ultrasound gel or high viscosity ultrasound gel. A suitable acoustic gel 50 may have a higher viscosity and higher surface tension than other unsuitable solutions such as water. The acoustic gel 50 may be evacuated of air or other gases to provide a clear conduit for acoustic transmission. Air or other gases may make up as little of the composition of the acoustic gel 50 as possible.

For example, in some embodiments, the acoustic gel 50 may be a semi-solid material which retains its shape when unconstrained by the wall 12. The acoustic gel 50 may absorb water or another low-viscosity fluid to form an acoustic pathway through the acoustic gel 50 which will not alter or distort shockwaves travelling through the acoustic gel. In such embodiments, fluid may be included within the chamber 26 to prevent infiltration of gas into the acoustic gel 50. Water or another fluid may be applied to the acoustic gel 50 during use to maintain the acoustic pathway and to prevent infiltration of gas into the acoustic gel 50.

FIG. 2 illustrates another possible embodiment of the acoustic gel pack 10. In some embodiments, the first pad 18 may be peeled off the first opening 14 in a single motion from a first side of the acoustic gel pack 10 to a second side. However, in some embodiments, the first pad 18 may include a perforation 24 to control the direction of the removal of the first pad 18. The perforation 24 may be any feature of the first pad 18 which allows separation and removal of the first pad 18 along a guided path. Examples of the perforation 24 may include indentations, micro-channels within the first pad 18, or a series punctures in the first pad 18. The perforation 24 may be arranged in a variety of shapes, such as a spiral, as shown in FIG. 2, or in a series of strips crossing consecutively across the wall 12 from the first side of the wall 12 to the second side of the wall 12. In some embodiments, the perforation 24, may extend only partially through the first pad 18 to prevent gas from infiltrating the acoustic gel 50 in the chamber 26. Similar arrangements may be made with the second pad 20.

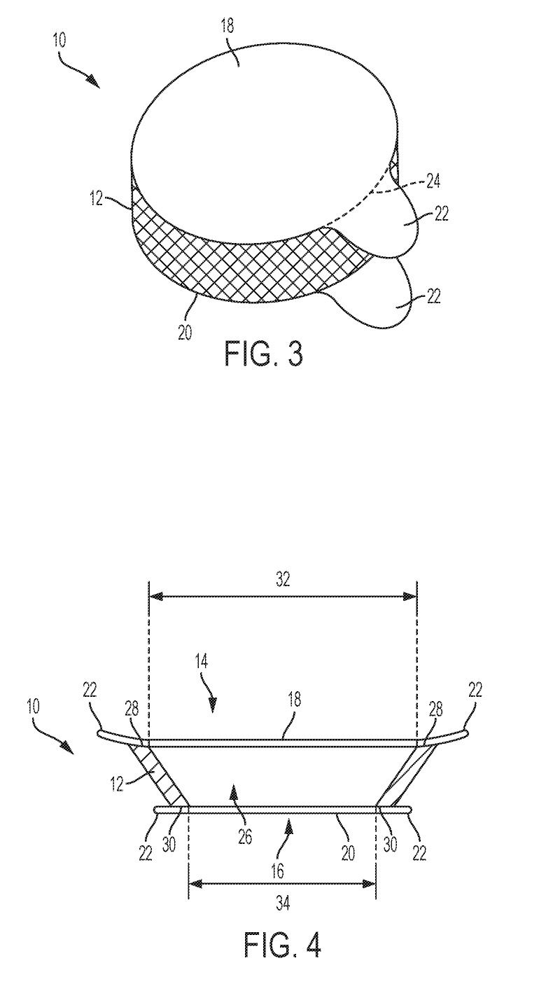

FIG. 3 illustrates another possible example of the acoustic gel pack 10. As shown in FIG. 1, in some embodiments, the tab 22 of the first pad 18 and the tab 22 of the second pad 20 may be on opposing sides of the acoustic gel pack 10 such that the first pad 18 may be removed, the acoustic gel pack 10 may be flipped and applied to the operating surface 64 and then the second pad 20 may be removed from the same orientation. Alternatively, FIG. 3 illustrates an example where the tab 22 of the first pad 18 is circumferentially aligned with the tab 22 of the second pad 20 such that flipping of the acoustic gel pack 10 need not occur.

Additionally, in some embodiments, the first pad 18 may be integrally coupled to the wall 12 such that when the first pad 18 is removed, the entire wall 12 is removed from the acoustic gel 50 as well. Such an arrangement may minimize the steps needed to quickly apply the acoustic gel 50. A similar configuration may be made with the second pad 20 and the wall 12.

FIG. 4 illustrates another possible embodiment of the acoustic gel pack 10. In some embodiments, the first side of the chamber 26 may have a first width 32 defined by the wall 12 which is greater than a second width 34 of the second side of the chamber 26 defined by the wall 12. As a consequence of this difference in width, the acoustic gel 50 may have a great width near the first opening 14 than near the second opening 16. With such an arrangement, the first pad 18 may be removed first and the first opening 14 may be applied to the operating surface 64. The acoustic gel 50 having greater width at or near the first opening 14 to allow the acoustic gel 50 to better maintain its shape on the operating surface 64. Such an arrangement may also allow the second pad 20 and the wall 12 to be removed more easily once the first opening 14 has been applied to the operating surface 64. Alternatively, the second width 34 of the second opening 16 may be greater than the first width 32 such that the acoustic gel 50 has a greater width at or near the wave head 62.

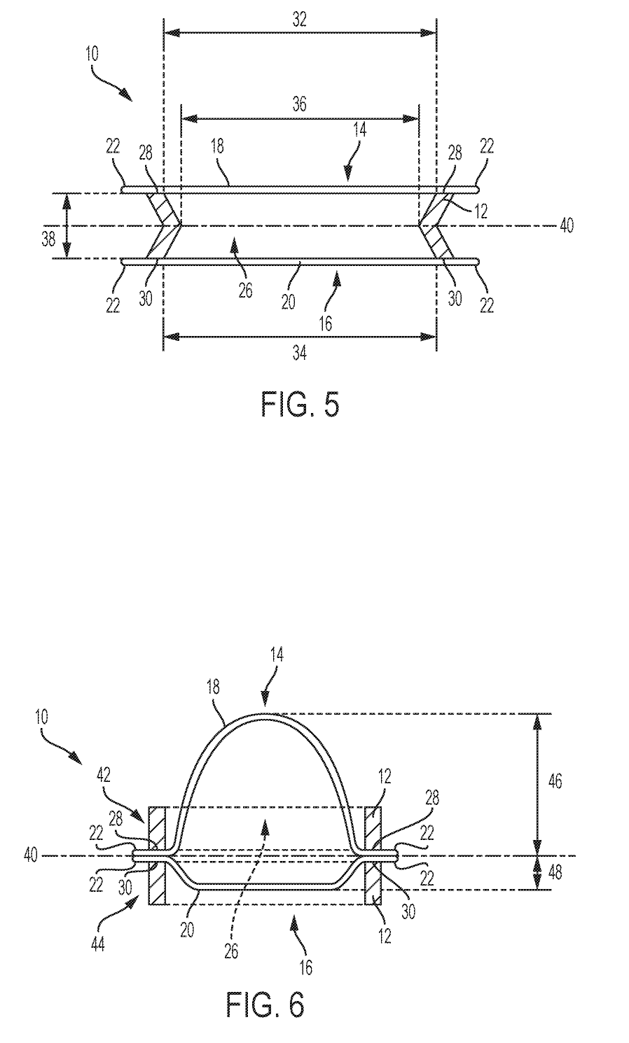

As illustrated in FIG. 5, in some embodiments, the first width 32 of the chamber 26 may be equal to the second width 34 of the chamber 26. Between the first opening 14 and the second opening 16, an intermediate width 36 of the chamber 26 may be less than either the first width 32 or the second width 34. In some embodiments, the intermediate width 36 may be located at a centerline 40 of the acoustic gel pack 10, midway between the first opening 14 and the second opening 16. The decreasing width of the chamber 26 defined by the wall may allow the wall 12 be more rigid and to better contain the acoustic gel 50 within the wall 12 when the acoustic gel pack 10 is applied to the operating surface 64. For example, in some embodiments, particularly where the acoustic gel pack 10 is significantly wider than the wave head (62 in FIG. 11), the wave head 62 may be inserted into and moved within the chamber 26 of the acoustic gel pack 10. To prevent the acoustic gel 50 from excessively spreading and thinning on the operating surface 64, the wall 12 may remain in place to contain the acoustic gel 50 within the chamber 26. The wall 12 may be configured to maintain a thickness 38 of the acoustic gel 50 within the chamber 26 at a sufficient level.

The wave head 62 may be any device which, when coupled with a lithotripsy machine (60 in FIG. 11), may transmit shockwaves from the lithotripsy machine through the wave head 62. Examples of the wave head may include a membrane or a drum. Shockwaves may any wave of acoustic energy which may be transmitted through an acoustic medium, such as the body of the patient, or the acoustic gel 50. Examples of shockwaves may include any acoustic waves, such as ultrasound burst waves, ultrasound waves, and ultrasound imaging waves.

FIG. 6 illustrates an alternative embodiment of the acoustic gel pack 10 including a wall 12 which is divided into a first portion 42 and a second portion 44. In such an embodiment, the first pad 18 and the second pad 20 may be sealed against each other, which the first portion 42 and second portion 44 of the wall holding the first pad 18 and second pad 20 together. To unseal the chamber 26, at least one of the first portion 42 or second portion 44 of the wall 12 much be removed from the respective first pad 18 or second pad 20. In some embodiments, the first pad 18 may be sealed to the second pad 20 with an adhesive.

In such an embodiment, the chamber 26 may be entirely defined by the shape of the first pad 18 and the second pad 20 which may be joined at the centerline 40 of the acoustic gel pack 10. The chamber 26 may be subdivided between a first portion defined by the first pad 18 and a second portion defined by the second pad 20. In some embodiments, a thickness 46 of the first portion of the chamber 26 may be equal or similar to a thickness 48 of the second portion of the chamber 26. As illustrated in FIG. 6, in some embodiments, the thickness 46 of the first portion may be greater than the thickness 48 of the second portion of the chamber 26. In such configurations, the first pad 18 may be large in size than the second pad 20 and may be shaped to contain more acoustic gel 50 within the chamber 26. For example, the first pad 18 may have a mound-like or pyramidal shape. The second pad 20 may have less thickness 48 but may have a greater width, operating as the base of the acoustic gel 50 when applied to the operating surface 64. The first pad 18 may have an outwardly convex curved surface. The second pad 20 may also have an outwardly convex curved surface which may be less convexly curved than the first pad 18.

In such an arrangement, the second portion 44 of the wall 12 may be removed, followed by the second pad 20, exposing the second opening 16 of the acoustic gel pack 10. The second opening 16 may then be applied to the operating surface 64. The first portion 42 of the wall 12 and the first pad 18 may then be removed. The pyramidal shape of the remaining acoustic gel 50 may then spread evenly as the wave head 62 is then applied. Where the thickness of the acoustic gel 50 is sufficient, the position of the wave head 62 with respect to the operating surface 64 may be adjusted without excessively thinning the acoustic gel 50 or introducing gas into the acoustic gel 50.

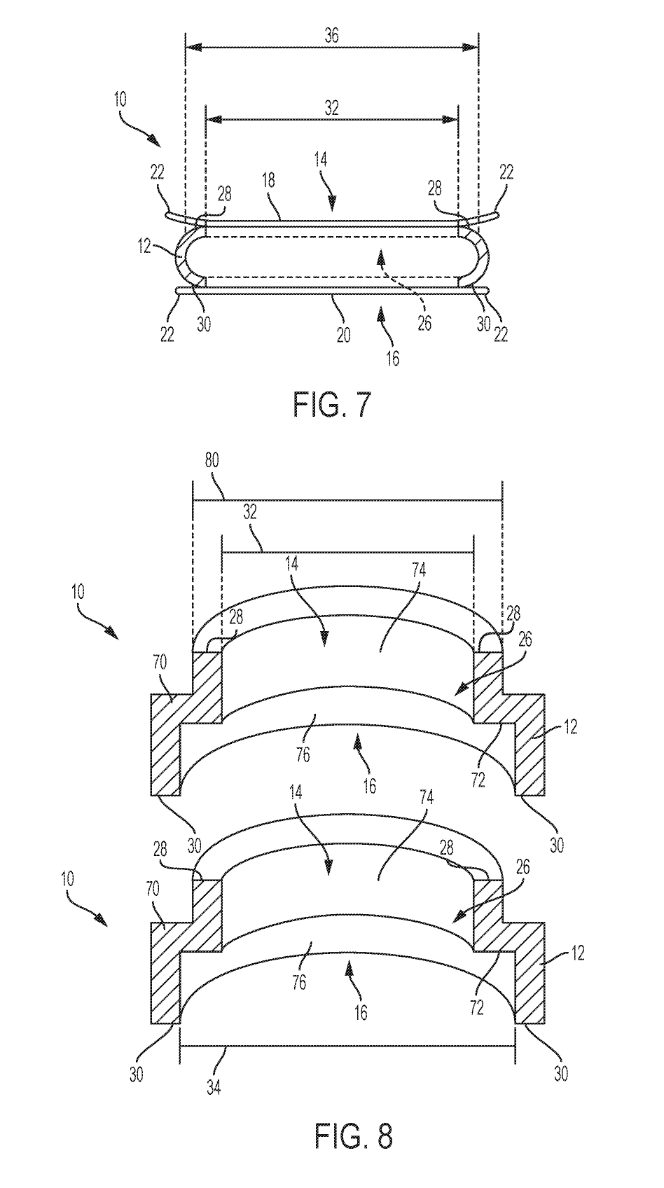

FIG. 7 illustrates another possible embodiment of the acoustic gel pack 10 having an intermediate width 36 which is greater than the width 32 of the chamber 26 at the first opening 14 or the thickness 34 of the chamber 26 at the second opening 16. In such an embodiment, the first opening 14 may be sized to closely encircle the wave head 62. The diameter 32 of the first opening 14 may be substantially similar to a diameter of the wave head 62. For example, the diameter 32 of the first opening 14 may less than 10% greater than the diameter of the wave head 62, but may be larger or smaller depending on the specific embodiment of the wave head 62. In some embodiments, the wall 12 encircling the first opening 14 may be friction fit against the wave head 62. The first pad 18 may be removed to expose the first opening 14. The acoustic gel pack 10 may then be applied to the wave head 62 through the first opening 14. The second pad 20 may then be removed to expose the second opening 16. The wave head 62 and acoustic gel pack 10 may then be applied to the operating surface 64. The additional acoustic fluid contained in the intermediate width 36 of the wall 12 may act as a reserve which may allow the wave head 62 and acoustic gel pack 10 to be moved on the operating surface 64.

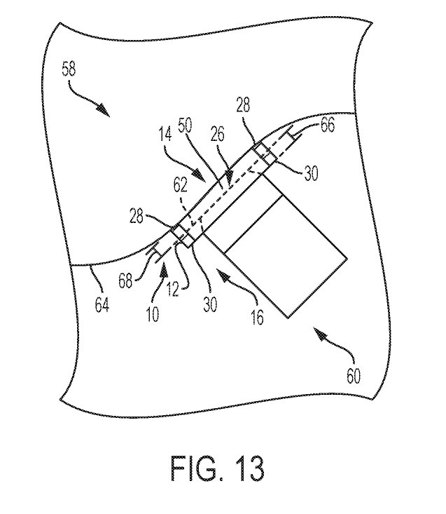

FIG. 8 illustrates another possible embodiment of the acoustic gel pack 10 which is stackable. In some procedures, the distance (68 in FIG. 13) through the acoustic gel 50 between the operating surface 64 and the wave head 62 may be important to direct the energy of the wave head at 62 a specific focal point below the operating surface 64. Depending on the patient (58 in FIG. 11), this distance may be unpredictable and vary considerably. Therefore, in some embodiments, the acoustic gel pack 10 may be stacked upon other acoustic gel packs 10 to provide a continuous pathway of acoustic gel 50 between the wave head 62 and the operating surface 64. The first surface 28 may include a upper shoulder 70 which is vertically offset below the first opening 14. The upper shoulder 70 may be any surface which is configured to receive the second surface 30 of another acoustic gel pack 10. The upper shoulder 70 may be spaced radially outward from the portion of the first surface 28 which is aligned with the first opening 14.

The second surface 30 may also include a lower shoulder 72 which is vertically offset below the second opening 16. The lower shoulder 72 may be any surface which is configured to receive the first surface 28 of another acoustic gel pack 10. The lower shoulder 72 may be spaced radially inward from the portion of the second surface 30 which is aligned with the second opening 16.

The presence of the upper shoulder 70 and lower shoulder 72 may divide the chamber 26 into an upper portion 74 and a lower portion 76. The upper portion 74 of the chamber 26 may have the width 32 of the first opening 14. The lower portion 76 of the chamber 26 may have the width 34 of the second opening 16 and may be greater than the width 32 of the first opening 14. The width 34 of the second opening 16 may be greater than a width 80 of the portion of the first surface 28 which is vertically aligned with the first opening 14. When the acoustic gel packs 10 are stacked upon one another, the total thickness of the acoustic gel may be greater than the combined thickness of the acoustic gel packs 10, creating a reserve of acoustic gel 50 which may be utilized during the procedure.

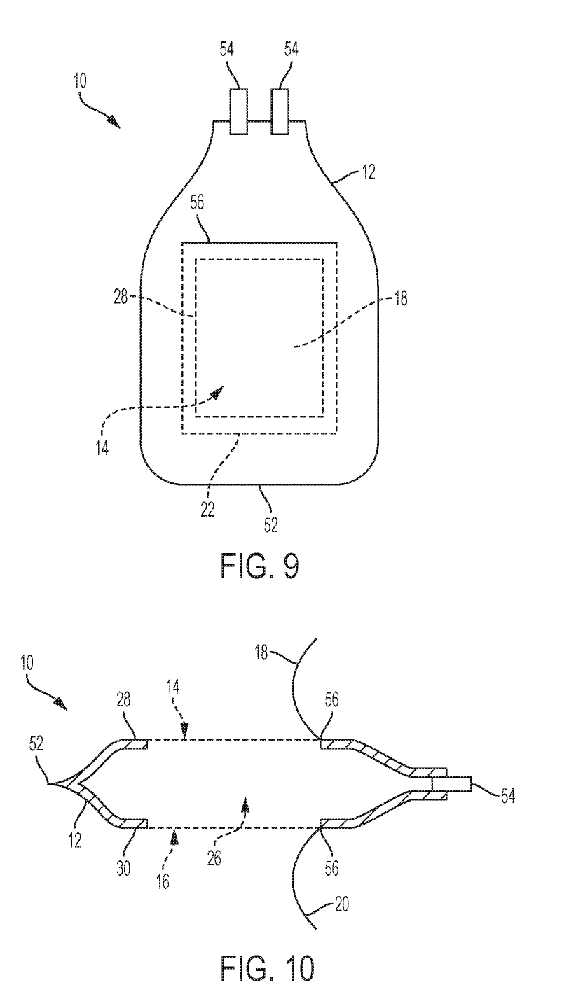

FIGS. 9 and 10 illustrate another possible configuration of the acoustic gel pack 10 in the form of a sealable bag. The first surface 28 and second surface 30 may meet to form a perimeter 52 of the sealable bag. The perimeter 52 may include one or more ports 54 usable to fill the bag with acoustic gel 50. The first pad 18 may be sealed to the first surface 28 in the form of a flap foldable at a joint 56. Similarly, the second pad 20 may be sealed to the second surface 30 in the form of a flap foldable at a joint 56. The first opening 14 and second opening 16 may be sized to receive the wave head 62.

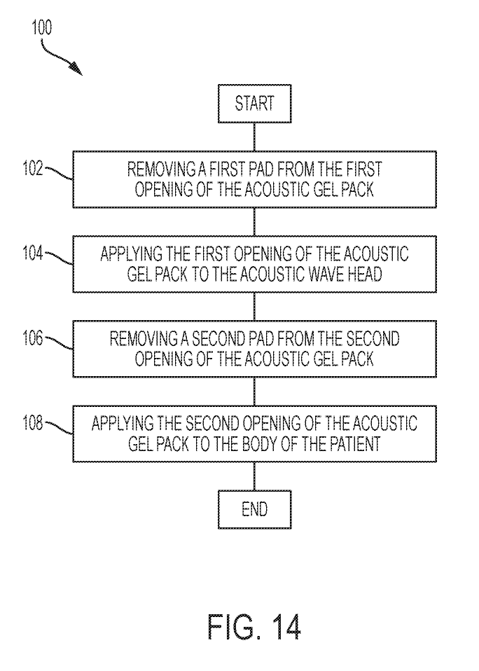

FIG. 14 illustrates a flow diagram of an example of a method (100) of performing lithotripsy using the acoustic gel pack 10. The steps may include additional, different, or fewer operations than illustrated in FIG. 14. The steps may be executed in a different order than illustrated in FIG. 14.

The first pad 18 is removed from the first opening 14 of the acoustic gel pack 10 (102). As illustrated in FIG. 11, the first opening 14 may also be arranged to face the operating surface 64 of the patient 58 before or after the first pad 18 is removed. Once the first pad 18 has been removed from the first opening 14, the first opening 14 of the acoustic gel pack 10 may then be applied to one of the wave head 62 or the operating surface 64 of the patient 58 (104). As described above, in some embodiments, particularly where the acoustic gel pack 10 is significantly wider than the wave head 62, the acoustic gel pack 10 may be applied to the operating surface 64. Where the acoustic gel pack 10 has a width which is close to the width of the wave head 62, the acoustic gel pack 10 may be applied to the wave head 62. Where the first surface 28 has an adhesive, the adhesive may be used to more securely couple the acoustic gel pack 10 to either of the operating surface 64 or the wave head 62.

The second pad 20 may be removed from the second opening 16 of the acoustic gel pack 10 (104). As illustrated in FIG. 12, removal of the first pad 18 and the second pad 20 may create a clear acoustic passage through the acoustic gel pack 10 from the first opening 14 to the second opening 16.

After the second pad 20 has been removed (104), the second opening 16 may be applied to the other of the operating surface 64 or the wave head 62 (106). Where the first opening 14 was first applied to the wave head 62, the wave head 62 and acoustic gel pack 10 combination may be advanced against the operating surface 64 of the patient 58. Alternatively, as illustrated in FIG. 13, where the first opening 14 was first applied to the operating surface 64 of the patient 58, the wave head 62 may be advanced into the acoustic gel 50 through the second opening 16.

Once coupled, the operating surface 64, the acoustic gel 50, and the wave head 62 may form an acoustic pathway to transmit shockwaves into the body to perform a lithotripsy procedure. Shockwaves created by a lithotripsy machine 60 may travel through the wave head 62, through the acoustic gel 50, across the operating surface 64, and through the body to break up objects at the focal point of the shockwaves. The distance 68 between the wave head 62 and the operating surface 64 may define the focal point of the shockwaves within the body of the patient 58. The focal point may be adjusted by changing a depth 66 at which the wave head 62 is submerged into the acoustic gel 50. The lateral position of the focal point may be adjusted by laterally moving the wave head 62 or by changing the angle of the wave head with respect to the operating surface 64. The lateral movement of the wave head 62 may be through the acoustic gel pack 10, across the acoustic gel 50, or may move along with the acoustic gel pack 10.

In some embodiments, the acoustic gel pack 10 may be filled with acoustic gel 50 before using the acoustic gel pack 10 with the wave head 62, such as during the manufacturing process. The acoustic gel pack 10 may be filled through the port 54 or may be filled where one of the first pad 18 or second pad 20 is not sealed to the first opening 14 or second opening 16. Once the chamber 26 has been filled, the first opening 14 or second opening 16 may be sealed, pushing out any extra gas. Some acoustic gels 50 require significant time to be infiltrated by gases, so the acoustic gel pack 10 may be slightly overfilled in an open air environment. Once the acoustic gel pack 10 is overfilled, the first pad 18 or second pad 20 may be applied, pushing out excess acoustic gel 50 to remove any air from the chamber 26.

Each component may include additional, different, or fewer components. For example, in some embodiments a port 54 may not be present. Additionally, in some embodiments, tabs 22 may not be present on the first pad 18 or second pad 20. Instead, the first pad 18 and second pad 20 may be punctured to unseal the first opening 14 and second opening 16.

The method (100) may be implemented with additional, different, or fewer components. For example, in some embodiments of the method (100) the step removing the first pad 18, removing the second pad 20, and removing the wall 12 may occur simultaneously and in a single action. Specifically, where the acoustic gel 50

The logic illustrated in the flow diagrams may include additional, different, or fewer operations than illustrated. The operations illustrated may be performed in an order different than illustrated.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed.

While various embodiments have been described, it will be apparent to those of ordinary skill in the art that many more embodiments and implementations are possible. Accordingly, the embodiments described herein are examples, not the only possible embodiments and implementations.

The subject-matter of the disclosure may also relate, among others, to the following aspects:
1. An acoustic gel pack, comprising:

a container configured to contain acoustic gel, the container comprising a wall, a first end having a first opening, and a second end having a second opening, wherein a chamber is defined within the wall between the first end and the second end;

a first pad removeably coupled to the container to seal the first opening; and

a second pad removeably coupled to the container to seal the second opening, wherein the seals formed by the first pad over the first opening and the second pad over the second opening are adapted to prevent fluid infiltration into the chamber.
2. The acoustic gel pack of aspect 1, wherein the wall comprises a collar encircling the chamber.
3. The acoustic gel pack of aspects 1 to 2, wherein the first opening has a width which is greater than a width of the second opening.
4. The acoustic gel pack of aspects 1 to 2, wherein the first opening has a first width, the second opening has a second width, and an intermediate width at a location within the chamber between the first opening and the second opening, wherein the intermediate width is less than both the first width and the second width.
5. The acoustic gel pack of aspects 1 to 4, wherein the collar further comprises a first surface at the first opening, wherein the first surface comprises an adhesive.
6. The acoustic gel pack of aspect 5, wherein the adhesive of the first surface of the collar is disposed to be exposed when the first pad is removed.
7. The acoustic gel pack of aspect 1, wherein the container comprises a bag, the bag comprising a top, a bottom, a first side, and a second side, wherein the first pad is removeably coupled to the first side of the bag and the second pad is removeably coupled to the second side of the bag.
8. The acoustic gel pack of aspects 1 to 7, wherein at least a portion of the first pad is coupled to the first side at a joint.
9. The acoustic gel pack of aspects 1 to 8, wherein the container comprises a first surface shaped to stackably receive a second surface of another container.
10. A lithotripsy system comprising:

an acoustic wave generating apparatus comprising an acoustic wave head; a container comprising a wall, the wall having a first opening and a second opening, wherein a chamber is defined within the wall between the first opening and the second opening, the chamber contains acoustic gel, and the second opening is sized to receive the acoustic wave head;

a first pad removeably coupled to the container to seal the first opening; and

a second pad removeably coupled to the container to seal the second opening.
11. The lithotripsy system of aspect 10, wherein the second opening of the container has a diameter which is substantially similar to a diameter of the acoustic wave head.
12. The lithotripsy system of aspects 10 to 11, wherein the first opening has a cross-sectional area which is greater than a cross-sectional area of the acoustic wave head.
13. The lithotripsy system of aspects 10 to 12, wherein the wall contains a first portion which is separable from a second portion, wherein the first pad and second pad are constrained between the first portion of the wall and the second portion of the wall.
14. The lithotripsy system of aspects 10 to 13, wherein a portion of the first pad is transparent material.
15. The lithotripsy system of aspects 10 to 14, wherein the chamber has a pyramidal shape.
16. The lithotripsy system of aspect 15, wherein the first pad has an outward convex curved surface.
17. A method of performing lithotripsy, comprising:

removing a first pad from a first opening of an acoustic gel pack, the acoustic gel pack comprising a wall, a first end having the first opening, and second end having a second opening, wherein a chamber is defined within the wall between the first opening and the second opening, the chamber containing air-evacuated acoustic gel;

applying the first opening of the acoustic gel pack to one of an acoustic wave head or a body of a patient;

removing a second pad from the second opening of the acoustic gel pack; and

applying the second opening of the acoustic gel pack to the other of the acoustic wave head or the body of the patient.
18. The method of aspect 17, further comprising moving the acoustic wave head within the wall of the acoustic gel pack.
19. The method of aspects 17 to 18, further comprising removing the wall from the body of the patient before applying the second opening of the acoustic gel pack to the other of the acoustic wave head or the body of the patient.
20. The method of aspects 17 to 19, further comprising adhesively coupling a surface of the wall of the acoustic gel pack to the body of the patient.

What is claimed is:

1. An acoustic gel pack, comprising:
a container configured to contain acoustic gel, the container comprising a wall, a first end having a first opening, and a second end having a second opening, wherein a chamber is defined within the wall between the first end and the second end;
a first pad removeably coupled to the container to seal the first opening; and
a second pad removeably coupled to the container to seal the second opening, wherein the seals formed by the first pad over the first opening and the second pad over the second opening are adapted to prevent fluid infiltration into the chamber.

2. The acoustic gel pack of claim 1, wherein the wall comprises a collar encircling the chamber.

3. The acoustic gel pack of claim 2, wherein the first opening has a width which is greater than a width of the second opening.

4. The acoustic gel pack of claim 2, wherein the first opening has a first width, the second opening has a second width, and an intermediate width at a location within the chamber between the first opening and the second opening, wherein the intermediate width is less than both the first width and the second width.

5. The acoustic gel pack of claim 2, wherein the collar further comprises a first surface at the first opening, wherein the first surface comprises an adhesive.

6. The acoustic gel pack of claim 5, wherein the adhesive of the first surface of the collar is disposed to be exposed when the first pad is removed.

7. The acoustic gel pack of claim 1, wherein the container comprises a bag, the bag comprising a top, a bottom, a first side, and a second side, wherein the first pad is removeably coupled to the first side of the bag and the second pad is removeably coupled to the second side of the bag.

8. The acoustic gel pack of claim 7, wherein at least a portion of the first pad is coupled to the first side at a joint.

9. The acoustic gel pack of claim 1, wherein the container comprises a first surface shaped to stackably receive a second surface of another container.

10. A lithotripsy system comprising:
an acoustic wave generating apparatus comprising an acoustic wave head;
a container comprising a wall, the wall having a first opening and a second opening, wherein a chamber is defined within the wall between the first opening and the second opening, the chamber contains acoustic gel, and the second opening is sized to receive the acoustic wave head;
a first pad removeably coupled to the container to seal the first opening; and
a second pad removeably coupled to the container to seal the second opening.

11. The lithotripsy system of claim 10, wherein the second opening of the container has a diameter which is substantially similar to a diameter of the acoustic wave head.

12. The lithotripsy system of claim 10, wherein the first opening has a cross-sectional area which is greater than a cross-sectional area of the acoustic wave head.

13. The lithotripsy system of claim 10, wherein the wall contains a first portion which is separable from a second portion, wherein the first pad and second pad are constrained between the first portion of the wall and the second portion of the wall.

14. The lithotripsy system of claim 10, wherein a portion of the first pad is transparent material.

15. The lithotripsy system of claim 10, wherein the chamber has a pyramidal shape.

16. The lithotripsy system of claim 15, wherein the first pad has an outward convex curved surface.

17. A method of performing lithotripsy, comprising:
removing a first pad from a first opening of an acoustic gel pack, the acoustic gel pack comprising a wall, a first end having the first opening, and second end having a second opening, wherein a chamber is defined within the wall between the first opening and the second opening, the chamber containing air-evacuated acoustic gel;
applying the first opening of the acoustic gel pack to one of an acoustic wave head or a body of a patient;
removing a second pad from the second opening of the acoustic gel pack; and
applying the second opening of the acoustic gel pack to the other of the acoustic wave head or the body of the patient.

18. The method of claim 17, further comprising moving the acoustic wave head within the wall of the acoustic gel pack.

19. The method of claim 17, further comprising removing the wall from the body of the patient before applying the second opening of the acoustic gel pack to the other of the acoustic wave head or the body of the patient.

20. The method of claim 17 further comprising adhesively coupling a surface of the wall of the acoustic gel pack to the body of the patient.
